(19) United States
(12) Patent Application Publication
(10) Pub. No.: US 2022/0175867 A1
(43) Pub. Date: Jun. 9, 2022

(54) GRAPE EXTRACT, METHOD FOR PRODUCING GRAPE EXTRACT LIQUID, METHOD FOR PRODUCING EXTRACTED AND PURIFIED PRODUCT OF GRAPE, AND METHOD FOR PRODUCING DRIED PRODUCT OF EXTRACTED AND PURIFIED PRODUCT OF GRAPE

(71) Applicant: ZEON CORPORATION, Tokyo (JP)

(72) Inventor: Naomi NISHIZAWA, Tokyo (JP)

(73) Assignee: ZEON CORPORATION, Tokyo (JP)

(21) Appl. No.: 17/456,605

(22) Filed: Nov. 26, 2021

(30) Foreign Application Priority Data
Dec. 7, 2020 (JP) 2020-203032

(51) Int. Cl. A61K 36/87

(52) U.S. Cl. CPC A61K 36/87; A61K 2236/333; A61K 2236/15; A61K 2236/37; A61K 2236/331

(57) ABSTRACT

Disclosed is a method for producing a grape extract which comprises extracting a grape at an extraction temperature of 15° C. or higher and 50° C. or lower using an extractant containing a polar solvent and an acid component. Also disclosed is a grape extract which comprises malvidin-3,5-diglucoside and a saccharide, wherein a dry mass content of the malvidin-3,5-diglucoside is 3% by mass or more, and a dry mass content of the saccharide is 80% by mass or less.

### TECHNICAL FIELD

The present disclosure relates to a grape extract, a method for producing a grape extract liquid, a method for producing an extracted and purified product of a grape, and a method for producing a dried product of an extracted and purified product of a grape.

### BACKGROUND

Anthocyanins are known to exhibit various physiological activities, such as visual impairment improving effects, antioxidant effects, vascularization effects, anti-inflammatory effects, and anti-tumor effects. As such, attempts have been made to utilize biological resources containing anthocyanins.

For example, blueberry fruit extracts have been reported to be used as anti-cancer agents (PTL 1) and compositions for improving brain function (PTL 2). Attempts have also been made to suppress the deposition of amyloid β protein by anthocyanins contained in bilberries (NPL 1).

### CITATION LIST

### Patent Literature

PTL 1: JP2003277271A

PTL 2: JP2007145825A

### Non-Patent Literature

NPL 1: MY. Yoshida, et al. “Anthocyanin suppresses the toxicity of AP deposits through diversion of molecular forms in vitro and in vivo models of Alzheimer's disease” Nutritional Neuroscience, 2016, Vol. 19(1), p. 32-42

### SUMMARY

In Japan, the market for drugs for treating dementia continues to expand drastically as the number of patients with this disease has been increasing due to the aging population. The market size is expected to expand by 1.75 times in 2022 compared with 2013. Generally, medical treatments for dementia entail side effects and other adverse events, which impose a large burden on patients. On the other hand, substances derived from biological resources are safe with fewer side effects and hence are suitable for daily or continuous ingestion. Thus, there are high expectations for substances that are derived from biological resources and exhibit neuronal function modulating effects.

As to anthocyanins, as mentioned above, blueberries and bilberries have been mainly studied as biological resources, and grapes have not been sufficiently studied.

The inventor focused on and studied grapes as a biological resource and established that a grape extract that comprises malvidin-3,5-diglucoside having the formula (1) shown below is useful as a raw material of food compositions or pharmaceutical compositions.

Malvidin-3,5-diglucoside in the grape extract may be in the form having a counter anion, e.g., chloride. There are no particular limitations on the parts and conditions of grapes used for extraction as a biological resource; a grape root, stem, leaf or fruit (pericarp, pulp or seed) may be used. A grape pomace or the like generated when making wine or the like may also be used.

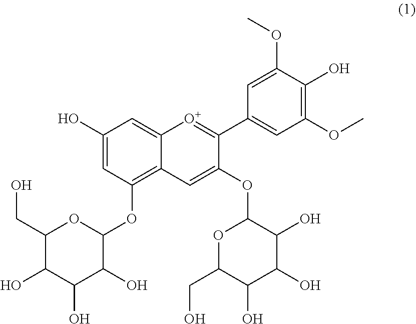 [SMILES: COc1cc(-c2[o+]c3cc(O)cc(OC4OC(CO)C(O)C(O)C4O)c3cc2OC2OC(CO)C(O)C(O)C2O)cc(OC)c1O]

Extensive studies conducted by the inventor, however, revealed that malvidin-3,5-diglucoside may not be extracted sufficiently depending on the extraction conditions. Thus, a need exists in the art to establish a technique that allows malvidin-3,5-diglucoside to be extracted in high yields from grapes in order for grape extracts containing malvidin-3,5-diglucoside to be used as a raw material for food compositions, pharmaceutical compositions or other compositions.

Further studies conducted by the inventor also revealed that while the use of a specific extraction method allows malvidin-3,5-diglucoside to be extracted in high yields from grapes, a dried powder obtained by drying the extract liquid obtained by the extraction method easily solidifies due to moisture absorption and becomes difficult to handle.

Accordingly, it would be helpful to provide a method for extracting malvidin-3,5-diglucoside from a grape in high yields.

It would also be helpful to provide a grape extract that, even when converted into dry powder form, does not easily solidify and has excellent handleability.

The inventor conducted extensive studies with the aim of solving the problems set forth above. As a result, the present inventor established that malvidin-3,5-diglucoside can be extracted from a grape in high yields by performing extraction in a specific extraction temperature range using a specific extractant. The inventor also established that a grape extract whose malvidin-3,5-diglucoside content in dry mass of the grape extract has a specific value or more and whose saccharide content in dry mass of the grape extract has a specific value or less does not easily solidify even when converted into dry powder form, and that performing purification after extraction can reduce the saccharide content while increasing the malvidin-3,5-diglucoside content in the extract. The inventor thus completed the present disclosure.

The present disclosure is intended to advantageously solve the above problems, and the disclosed grape extract comprises malvidin-3,5-diglucoside and a saccharide, wherein a malvidin-3,5-diglucoside content in dry mass of the grape extract is 3% by mass or more, and a saccharide content in dry mass of the grape extract is 80% by mass or less. When the malvidin-3,5-diglucoside content is equal to or higher than the lower limit value as described above, the grape extract can be advantageously used as a raw material for food compositions, pharmaceutical compositions or other compositions. When the saccharide content is equal to or lower than the upper limit value as described above, solidification due to moisture absorption can be suppressed even when the grape extract is converted into dry powder form.

The malvidin-3,5-diglucoside content and the saccharide content in dry mass of the grape extract can be measured using the method described in Examples.

Also, the present disclosure is intended to advantageously solve the problems set forth above, and the disclosed method for producing a grape extract liquid comprises extracting a grape at an extraction temperature of 15° C. or higher and 50° C. or lower using an extractant containing a polar solvent and an acid component. When extraction is performed in a specific extraction temperature range using a specific extractant as described above, it is possible to extract malvidin-3,5-diglucoside from a grape in high yields to provide a grape extract liquid having a high malvidin-3,5-diglucoside content.

In the disclosed method for producing a grape extract liquid, it is preferred that the polar solvent comprise at least one solvent selected from the group consisting of water, methanol, ethanol, isopropanol, butanol, propylene glycol, butylene glycol, glycerin, acetone, ethyl acetate, methyl ethyl ketone, dimethylformamide, dimethyl sulfoxide, and tetrahydrofuran. The use of one or more of these solvents allows malvidin-3,5-diglucoside to be well extracted from a grape.

In the disclosed method for producing a grape extract liquid, it is preferred that the extractant comprises at least one acid selected from the group consisting of formic acid, citric acid, lactic acid, malic acid, tartaric acid, succinic acid, hydrochloric acid, and phosphoric acid. The use of one or more of these acid components allows malvidin-3,5-diglucoside to be well extracted from a grape.

In the disclosed method for producing a grape extract liquid, it is preferred that the grape comprise a pericarp of one or more types of grapes selected from the group consisting of Vitis Coignetiae, Vitis flexuosa, a cross species between Vitis Coignetiae and Vitis flexuosa, a cross species between Vitis Coignetiae and Vitis Vinifera, and a cross species between Vitis flexuosa and Vitis Vinifera. This is because the pericarp of these types of grapes is rich in malvidin-3,5-diglucoside content and is specific in the composition of anthocyanins.

In the disclosed method for producing a grape extract liquid, it is preferred that the grape be crushed or squeezed. The use of a crushed or squeezed grape can further increase the yield of malvidin-3,5-diglucoside.

Also, the present disclosure is intended to advantageously solve the problems set forth above, and the disclosed method for producing an extracted and purified product of a grape comprises purifying a grape extract liquid produced using any of the methods for producing a grape extract liquid described above. When the grape extract liquid obtained by the above-described production method is purified, it is possible to obtain an extracted and purified product of a grape which does not easily solidify even when converted into dry powder form by reducing the saccharide content while increasing the malvidin-3,5-diglucoside content.

In the disclosed method for producing an extracted and purified product of a grape, it is preferred that the purifying comprises: a step (a) of bringing the grape extract liquid in contact with a resin; a step (b) of washing the resin with an acidic aqueous solution after step (a); and a step (c) of bringing the resin in contact with a mixed liquid of an acidic aqueous solution and an alcohol after step (b). When steps (a) to (c) are performed with a resin, it is possible to further reduce the saccharide content in the extracted and purified product of a grape while increasing the malvidin-3,5-diglucoside content.

In the disclosed method for producing an extracted and purified product of a grape, it is preferred that the resin comprise at least one resin selected from the group consisting of an aromatic resin, a substituted aromatic resin, and an acrylic resin. The use of one or more of these resins allows the grape extract liquid to be well purified.

In the disclosed method for producing an extracted and purified product of a grape, it is preferred that the acidic aqueous solution comprise at least one acid selected from the group consisting of formic acid, citric acid, lactic acid, malic acid, tartaric acid, succinic acid, hydrochloric acid, and phosphoric acid. The use of an acidic aqueous solution that comprises one or more of these acid components allows the grape extract liquid to be well purified.

Also, the present disclosure is intended to advantageously solve the problems set forth above, and the disclosed method for producing a dried product of an extracted and purified product of a grape preferably comprises drying an extracted and purified product of a grape produced using any of the methods for producing an extracted and purified product of a grape described above. When the extracted and purified product of a grape obtained by the above-described production method is dried, it is possible to obtain a dried product of an extracted and purified product of a grape which does not easily solidify.

According to the disclosed method for producing a grape extract liquid, it is possible to extract malvidin-3,5-diglucoside from a grape in high yields.

According to the disclosed method for producing an extracted and purified product of a grape, the disclosed method for producing a dried product of an extracted and purified product of a grape, and the disclosed grape extract, it is possible to provide a grape extract that, even when converted into dry powder form, does not easily solidify and has excellent handleability.

### DETAILED DESCRIPTION

The following provides a detailed description of embodiments of the present disclosure.

The disclosed grape extract can be produced for example using the disclosed method for producing an extracted and purified product of a grape and the disclosed method for producing a dried product of an extracted and purified product of a grape, and can be advantageously used as a raw material of food compositions, pharmaceutical compositions or other compositions. The disclosed method for producing a grape extract liquid can be used when extracting malvidin-3,5-diglucoside from a grape. The grape extract liquid obtained by the disclosed method for producing a grape extract liquid can be used as a raw material when obtaining an extracted and purified product of a grape by the disclosed method for producing an extracted and purified product of a grape. The extracted and purified product of a grape obtained by the disclosed method for producing an extracted and purified product of a grape can be used as a raw material when obtaining a dried product of an extracted and purified product of a grape by the disclosed method for obtaining a dried product of an extracted and purified product of a grape.

(Grape Extract) The disclosed grape extract comprises malvidin-3,5-diglucoside and a saccharide and may optionally further comprise a solvent. Specifically, the disclosed grape extract may be in the form of solution, dispersion or paste or may be in dry solid form.

The disclosed grape extract requires that the malvidin-3,5-diglucoside content in dry mass of the grape extract be 3% by mass or more and the saccharide content in dry mass of the grape extract be 80% by mass or less.

The disclosed the grape extract may further comprise anthocyanins other than malvidin-3,5-diglucoside (hereafter also referred to as “other anthocyanins), such as delphinidin-3-glucoside, malvidin-3-glucoside, malvidin-3-coumaroylglucoside, malvidin-3-coumaroylglucoside-5-glucoside, and resveratrol. Herein, these anthocyanins may be in the form having a counter anion, such as, for example, chloride.

<Malvidin-3,5-diglucoside>

Malvidin-3,5-diglucoside is a compound having the formula (1) shown above. Herein, malvidin-3,5-diglucoside may be in the form having a counter anion, such as, for example, chloride.

The proportion of malvidin-3,5-diglucoside in dry mass of the grape extract needs to be 3% by mass or more and is preferably 8% by mass or more, more preferably 10% by mass or more, and still more preferably 20% by mass or more. There are no particular limitations on the upper limit of the proportion of malvidin-3,5-diglucoside as long as it is less than 100% by mass. The proportion of malvidin-3,5-diglucoside is usually 50% by mass or less. When the proportion of malvidin-3,5-diglucoside is equal to or higher than the lower limit value, handling ease during processing or formulation improves and also costs of processing or formulation decrease, so that the grape extract can be advantageously used as a raw material of food compositions, pharmaceutical compositions or other compositions.

<Saccharides>

There are no particular limitations on saccharides included in the grape extract. Examples of saccharides include those contained in grapes, such as fructose and glucose.

The proportion of the saccharide in dry mass of the grape extract needs to be 80% by mass or less and is preferably 60% by mass or less, more preferably 50% by mass or less, still more preferably 40% by mass or less, and particularly preferably 20% by mass or less. There are no particular limitations on the lower limit of the proportion of the saccharide as long as it is more than 0% by mass. The proportion of the saccharide is usually 3% by mass or more. When the proportion of the saccharide is equal to or lower than the upper limit value, it is possible to suppress solidification caused by moisture absorption when the grape extract is converted into dry powder form.

<Solvent>

There are no particular limitations on the solvent that may be optionally included in the grape extract. Examples of solvents include aqueous solutions such as water and acidic aqueous solutions; organic solvents such as alcohols; or mixture solutions of two or more of the foregoing. Examples of acidic aqueous solutions include aqueous solutions of at least one acid selected from the group consisting of formic acid, citric acid, lactic acid, malic acid, tartaric acid, succinic acid, hydrochloric acid, and phosphoric acid. Examples of alcohols include methanol, ethanol, and propanol.

The solids concentration of the grape extract is not particularly limited. From the viewpoint of handling property, however, the solids concentration is preferably 5% by mass or more, and more preferably 10% by mass or more.

<Applications of Grape Extract>

The grape extract comprises malvidin-3,5-diglucoside and may have such effects as anti-aging, anti-stress, anti-fatigue, restoration/improvement of learning ability, restoration/improvement of memory ability, or restoration/improvement of reflection and reaction ability. Because the grape extract is of biological resource origin, it is safe with less side effects and is suitable for daily or continuous ingestion.

As such, the grape extract can be suitably used as a raw material when producing food compositions or pharmaceutical compositions. Examples of subjects to be administered with food compositions or pharmaceutical compositions include mammals, e.g., humans, domestic animals (e.g., horses, cows, pigs), pet animals (e.g., dogs, cats), and experimental (test) animals (e.g., mice, rats), with humans being preferred.

[Food Composition]

The food composition that comprises the grape extract described above can be a functional food, a dietary supplement, a health supplement, a nutritionally fortified food, a food with a functional claim, a food for special dietary use, a food for specified health use, or a food with a nutritional function.

The food composition can comprise ingredients acceptable in the production of foods. Examples thereof include carriers, excipients (e.g., lactose, starch, sucrose, dextrin, hydroxypropylcellulose, polyvinylpyrrolidone), disintegrating agents, buffers, emulsifiers, suspending agents, stabilizers, preservatives, solvents (e.g., water, saline, and organic solvents such as ethanol). Foods can also comprise proteins, carbohydrates, lipids, vitamins, minerals, organic acids, organic bases, flavors, and other ingredients.

The food composition can be in any form including solids, liquids, mixtures, suspensions, pastes, gels, powders, and capsules. The food composition can be a beverage, confectionery, seasoning, daily dish or processed food. The food composition may also be a supplement.

Diseases and conditions to be treated, administration regimen (subject, dose, period) etc. of the food composition can be determined depending on the purpose. Specifically, the food composition can be used for anti-aging, anti-stress, anti-fatigue, restoration/improvement of learning ability, restoration/improvement of memory ability, or restoration/improvement of reflection and reaction ability.

[Pharmaceutical Composition]

The pharmaceutical composition that comprises the grape extract described above can be a pharmaceutical or quasi-pharmaceutical agent.

The pharmaceutical composition can comprise a pharmaceutically acceptable ingredient. Examples thereof include carriers, excipients (e.g., lactose, starch, sucrose, dextrin, hydroxypropylcellulose, polyvinylpyrrolidone), disintegrating agents, buffers, emulsifiers, suspending agents, stabilizers, preservatives, solvents (e.g., water, saline, and organic solvents such as ethanol). The pharmaceutical composition can also comprise other pharmacological ingredients having a pharmacological activity.

The pharmaceutical composition can be used as a pharmaceutical or quasi-pharmaceutical agent. The pharmaceutical composition may be for oral or parenteral administration, but is preferably for oral administration. Dosage forms for oral administration can include powders, tablets, coated tablets, sugar-coated tablets, soft or hard capsules, solutions, emulsions, and suspensions. Dosage forms for parenteral administration can include suppositories, injections, infusions, ointments, creams, and gels.

Diseases and conditions to be treated, administration regimen (subject, dose, period) etc. of the pharmaceutical composition can be determined according to the purpose. Specifically, the pharmaceutical composition can be used for anti-aging, anti-stress, anti-fatigue, restoration/improvement of learning ability, restoration/improvement of memory ability, or restoration/improvement of reflection and reaction ability.

(Method for Producing Grape Extract Liquid) The disclosed method for producing a grape extract liquid is a method wherein a grape extract liquid that comprises malvidin-3,5-diglucoside is produced from a grape used as a raw material (material to be extracted), and the method comprises extracting a grape using an extractant containing a polar solvent and an acid component at an extraction temperature of 15° C. or higher and 50° C. or lower (extraction step). Note that the disclosed method for producing a grape extract liquid may include a pre-treatment step of pre-treating a grape before the extraction step. Further, the extract liquid obtained in the extraction step of the disclosed method for producing a grape extract liquid can be separated from the grape extract residue using any solid-liquid separation methods, such as filtration.

<Pre-treatment Step>

In the pre-treatment step, a grape is pre-treated so that malvidin-3,5-diglucoside can be well extracted from the grape in the extraction step.

There are no particular limitations on pre-treatment methods. The pre-treatment method can include at least one method selected from the group consisting of crushing, squeezing, and drying. Crushing can be performed using for example a mixer, squeezing can be performed using for example a fruit blister, and drying can be performed using for example a lyophilizer.

<Extraction Step>

In the extraction step, a grape is extracted using an extractant containing a polar solvent and an acid component at an extraction temperature of 15° C. or higher and 50° C. or lower. By performing extraction using the specific extractant in the specific extraction temperature range as described above, it is possible to extract malvidin-3,5-diglucoside from the grape in high yields.

[Grapes]

There are no particular limitations on the parts and condition of the material to be extracted as long as it is a grape. The material to be extracted can be at least one selected from the group consisting of a grape root, stem, leaf and fruit (pericarp, pulp or seed), a grape pomace or the like generated when making wine or the like, and so forth.

It is preferred that the material to be extracted comprise at least a grape pericarp because the grape pericarp is rich in anthocyanins, especially in malvidin-3,5-diglucoside. Because it is burdensome to separate only the pericarp from the fruit, the material to be extracted is preferably the fruit of a grape from the viewpoint of simplicity.

Any types of grapes can be used as the material to be extracted. For their high marvidin-3,5-diglucoside content and specificity of marvidin-3,5-diglucoside in the composition of anthocyanins, however, it is preferred to use one or more types of grapes selected from the group consisting of Vitis Coignetiae, Vitis flexuosa, a cross species between Vitis Coignetiae and Vitis flexuosa, a cross species between Vitis Coignetiae and Vitis Vinifera, and a cross species between Vitis flexuosa and Vitis Vinifera. It is more preferred to use the pericarp of one or more of these types of grapes.

[Extractant]

As the extractant, an extractant containing a polar solvent and an acid component can be used.

From the viewpoint that malvidin-3,5-diglucoside is well extracted from a grape, as the polar solvent, it is preferred to use at least solvent selected from the group consisting of water, methanol, ethanol, isopropanol, butanol, propylene glycol, butylene glycol, glycerin, acetone, ethyl acetate, methyl ethyl ketone, dimethylformamide, dimethyl sulfoxide, and tetrahydrofuran, it is more preferred to use water, methanol, ethanol, ethanol aqueous solution, dimethylformamide, dimethyl sulfoxide or tetrahydrofuran, and it is still more preferred to use water, ethanol or ethanol aqueous solution.

From the viewpoint that malvidin-3,5-diglucoside is well extracted from a grape, as the acid component, it is preferred to use at least one acid selected from the group consisting of formic acid, citric acid, lactic acid, malic acid, tartaric acid, succinic acid, hydrochloric acid, and phosphoric acid, it is more preferred to use formic acid, hydrochloric acid or citric acid, and it is still more preferred to use hydrochloric acid.

There are no particular limitations on the composition of the extractant as long as it is possible to perform extraction in the presence of the polar solvent and the acid component. From the viewpoint that malvidin-3,5-diglucoside is well extracted from a grape under acidic conditions, the concentration of the acid component in the extractant is preferably 0.000001 mol/L or more, more preferably 0.00001 mol/L or more, still more preferably 0.0001 mol/L or more, but is preferably 0.1 mol/L or less, more preferably 0.01 mol/L or less.

[Extraction Temperature]

The extraction temperature needs to be 15° C. or higher and 50° C. or lower and is preferably 20° C. or higher, more preferably 25° C. or higher, but is preferably 45° C. or lower, more preferably 30° C. or lower. When the extraction temperature is equal to or higher than the lower limit value, malvidin-3,5-diglucoside can be efficiently extracted in a short time. When the extraction temperature is equal to or lower than the upper limit value, it is possible to prevent thermal degradation of malvidin-3,5-diglucoside to increase its yield.

[Other Extraction Conditions]

There are no particular limitations on the extraction time. Extraction time can be 1 hour or more and 12 hours or less, for example.

Extraction can be performed using any extraction device, such as a Soxhlet extractor or a high-speed high-pressure extraction device.

<Composition of Extract Liquid>

The grape extract liquid obtained through the extraction step usually comprises malvidin-3,5-diglucoside and the extractant, and optionally further comprises a saccharide and/or other anthocyanins. Examples of saccharides and other anthocyanins include those described above as to the disclosed grape extract.

(Method for Producing Extracted and Purified Product of Grape)

The disclosed method for producing an extracted and purified product of a grape comprises purifying the grape extract liquid produced using the disclosed method for producing a grape extract liquid (purification step). Note that the grape extract liquid may be purified after concentrated using any concentration methods, e.g., concentration under reduced pressure. From the viewpoint of preventing degradation of malvidin-3,5-diglucoside, concentration is preferably performed at 50° C. or lower.

<Purification Step>

In the purification step, the grape extract liquid is purified using any purification methods capable of achieving at least one of increasing the malvidin-3,5-diglucoside content and/or removing the saccharide, whereby an extracted and purified product of a grape is obtained in which the saccharide content is reduced while increasing the malvidin-3,5-diglucoside content. When the material to be extracted to obtain the grape extract liquid contains a grape part that is rich in saccharides, e.g., pulp, the grape extract liquid may have a high saccharide content. However, performing such a purification step makes it possible to reduce the saccharide content.

There are no particular limitations on purification methods. The purification methods can include those that involve the use of a resin, e.g., purification methods that involve the use of a synthetic adsorption resin, and purification methods that involve the use of an ion exchange resin. Preferred are purification methods that involve the use of a synthetic adsorption resin.

Any types of resins can be used for purification as long as the resin may physically adsorb anthocyanins, including malvidin-3,5-diglycoside. Preferred are synthetic adsorption resins, with aromatic resins (e.g., styrene resins), substituted aromatic resins, and acrylic resins being more preferred, and hydrophobic synthetic adsorption resins with a polystyrene-divinylbenzene crosslink being further preferred. The synthetic adsorption resins usually do not have functional groups such as those of ion exchange resins; only one type alone can be used or two or more types can be used in combination.

Examples of aromatic resins or substituted aromatic resins include those commercially available under the following tradenames: DIAION HP20, DIAION HP21, Sepabeads SP850, Sepabeads SP825L, and Sepabeads SP700 (manufactured by Mitsubishi Chemical Corporation); Macronet MN250, PuroSorb PAD600, PuroSorb PAD550, PuroSorb PAD900, and PuroSorb PAD350 (manufactured by Purolite Corporation); and Amberlite FPX66 (manufactured by Rohm & HaaS Corporation).

Examples of acrylic resins include those commercially available under the following tradenames: PuroSorb PAD610, and PuroSorb PAD950 (manufactured by Purolite Corporation); and Amberlite XAD-7HP (manufactured by Rohm & HaaS Co., Ltd.).

It is preferred that resins that may be used for purification have a pore diameter of 40 Å or more and 700 Å or less from the viewpoint of adsorption capability.

It is also preferred that resins that may be used for purification have a specific surface area of 400 m²/g or more and 1,100 m²/g or less from the viewpoint of adsorption capability.

In the purification method using a resin, it is preferred to perform: a step (a) of bringing the grape extract liquid in contact with the resin; a step (b) of washing the resin with an acidic aqueous solution after step (a); and a step (c) of bringing the resin in contact with a mixed liquid of an acidic aqueous solution and an alcohol after step (b). By performing steps (a) to (c), it is possible to remove such unnecessary components as water-soluble pigments, saccharides, proteins and/or amino acids while selectively recovering anthocyanins, including malvidin-3,5-diglucoside.

[Step (a)]

There are no particular limitations on the type of the process whereby the grape extract liquid is brought in contact with the resin in step (a). The process may be of either batch type or continuous type that uses, for example, a column. Step (a) is preferably performed in a continuous process using a column packed with the resin.

There are no particular limitations on the manner in which the grape extract liquid is brought in contact with the resin in a batch process; 1 to 50 volumes of the grape extract liquid can be brought in contact with one volume of the resin for 1 hour to overnight.

There are no particular limitations on the manner in which the grape extract liquid is brought in contact with the resin in a continuous process; 1 to 50 volumes of the grape extract liquid can be brought in contact with one volume of the resin so that the space velocity (SV) is 1 to 5 h⁻¹.

From the viewpoint of selectively allowing anthocyanins, including malvidin-3,5-diglucoside, to be adsorbed on the resin, the temperature at which the grape extract liquid is brought into contact with the resin is preferably 5° C. or higher, more preferably 10° C. or higher, still more preferably 20° C. or higher, but is preferably 60° C. or lower, more preferably 50° C. or lower, and still more preferably 25° C. or lower.

[Step (b)]

In step (b), the resin is washed with an acidic aqueous solution to remove non-adsorbed components. The acidic aqueous solution preferably has a pH of 6 or less. As the acidic aqueous solution, it is preferred to use an aqueous solution that comprises at least one acid selected from the group consisting of formic acid, citric acid, lactic acid, malic acid, tartaric acid, succinic acid, hydrochloric acid, and phosphoric acid, it is more preferred to use an aqueous solution that comprise formic acid, hydrochloric acid or citric acid, and it is still more preferred to use an aqueous solution that comprises hydrochloric acid.

When the grape extract liquid has been brought into contact with the resin in a batch process in step (a), it is only necessary that the resin be washed with the acidic aqueous solution after separating the resin from the supernatant.

When the grape extract liquid has been brought into contact with the resin in a continuous process in step (a), the resin may be washed by passing the acidic aqueous solution through the resin so that the space velocity (SV) is about 5 h⁻¹.

From the viewpoint that the non-adsorbed components are well removed, the temperature at which the resin is washed with the acidic aqueous solution is preferably 5° C. or higher, more preferably 10° C. or higher, still more preferably 20° C. or higher, but is preferably 60° C. or lower, more preferably 50° C. or lower, and still more preferably 25° C. or lower.

[Step (c)]

In step (c), the washed resin is brought into contact with a mixed liquid of an acidic aqueous solution and an alcohol to liberate anthocyanins adsorbed on the resin. It is preferred that the mixed liquid has a pH of 6 or less. As the acidic aqueous solution, it is preferred to use an aqueous solution that comprises at least one acid selected from the group consisting of formic acid, citric acid, lactic acid, malic acid, tartaric acid, succinic acid, hydrochloric acid, and phosphoric acid, it is more preferred to use an aqueous solution that comprises formic acid, hydrochloric acid or citric acid, and it is still more preferred to use an aqueous solution that comprises hydrochloric acid. As the alcohol, it is preferred to use methanol, ethanol, isopropanol or butanol, and it is more preferred to use methanol or ethanol.

When step (c) is performed in a batch process, it is only necessary that the resin and the mixed liquid be brought into contact with each other for about 1 hour.

When step (c) is performed in a continuous process, it is only necessary that the mixed liquid be passed so that the space velocity (SV) is about 5 h⁻¹.

From the viewpoint of allowing anthocyanins, including malvidin-3,5-diglucoside, to be well liberated from the resin, the temperature at which the resin is washed with the acidic aqueous solution is preferably 5° C. or higher, more preferably 10° C. or higher, still more preferably 20° C. or higher, but is preferably 60° C. or lower, more preferably 50° C. or lower, and still more preferably 25° C. or lower.

<Composition of Extracted and Purified Product of Grape>

The extracted and purified product of a grape obtained by purifying the grape extract liquid is usually a liquid or pasty composition and comprises malvidin-3,5-diglucoside, a saccharide, and the solvent used for purification (e.g., mixed liquid of acidic aqueous solution and alcohol), and optionally further comprise other anthocyanins.

The extracted and purified product of a grape may correspond to the disclosed grape extract described above. Specifically, the malvidin-3,5-diglucoside content in dry mass of the extracted and purified product is preferably 3% by mass or more, more preferably 8% by mass or more, still more preferably 10% by mass or more, and particularly preferably 20% by mass or more. The saccharide content in dry mass of the extracted and purified product is preferably 80% by mass or less, more preferably 60% by mass or less, still more preferably 50% by mass or less, still more preferably 40% by mass or less, and particularly preferably 20% by mass or less.

(Method for Producing Dried Product of Extracted and Purified

Product of Grape)

The disclosed method for producing a dried product of an extracted and purified product of a grape comprises drying the extracted and purified product of a grape produced by the disclosed method for producing an extracted and purified product of a grape (drying step). The disclosed method for producing a dried product of an extracted and purified product of a grape may optionally comprise, prior to the drying step, performing pre-treatment (e.g., concentration) on the extracted and purified product of a grape.

<Drying Step>

The extracted and purified product of a grape can be dried using any drying methods. From the viewpoint of preventing degradation of malvidin-3,5-diglucoside, the extracted and purified product is preferably dried at a temperature of 50° C. or lower and is more preferably dried by lyophilization or spray drying.

<Composition of Dried Product of Extracted and Purified Product of Grape>

The dried product of an extracted and purified product of a grape, obtained by drying the extracted and purified product of a grape, is usually a solid composition such as a powder composition and comprises malvidin-3,5-diglucoside and a saccharide, and optionally further comprises other anthocyanins.

The dried product of the extracted and purified product of a grape may correspond to the disclosed grape extract described above. Specifically, the malvidin-3,5-diglucoside content in dry mass of the dried product is preferably 3% by mass or more, more preferably 8% by mass or more, still more preferably 10% by mass or more, and particularly preferably 20% by mass or more. The saccharide content in dry mass of the dried product is preferably 80% by mass or less, more preferably 60% by mass or less, still more preferably 50% by mass or less, still more preferably 40% by mass or less, and particularly preferably 20% by mass or less.

### EXAMPLES

Hereinafter, the present disclosure will be described in more detail with reference to Examples, which however shall not be construed as limiting the technical scope of the present disclosure.

In Examples and Comparative Examples, quantification of malvidin-3,5-diglucoside, glucose and fructose, and evaluation of solidification of grape extracts were performed by the methods described below.

<Quantification of Malvidin-3,5-diglucoside>

Quantification was carried out by high-performance liquid chromatography (HPLC) as follows:

Analysis was performed in a gradient system using two types of liquids as eluents: Liquid A (5% formic acid in ultrapure water) and liquid B (1:1 (by volume) mixture of methanol and acetonitrile) on the column ZORBAX SB-C18 (4.6 mm diameter×250 mm length, particle size: 3.5 μm) at a flow rate of 1 mL/min with UV detection at 550 nm. The gradient condition is as shown in Table 1 below. The ingredients were identified and quantified using a HPLC library and by comparison with the measured values of standards.

TABLE 1

| | Composition | |
|---|---|---|
| Time (min) | Liquid A | Liquid B |
| 0 | 100 | 0 |
| 30 | 25 | 75 |
| 35 | 0 | 100 |

<Quantification of Glucose and Fructose>

Quantification was carried out by high-performance liquid chromatography (HPLC) as follows:

Analysis was performed in a system using water and acetonitrile as eluents on the column HILIC pak VG-50 4E (4.6 mm diameter×250 mm length, particle size: 5 μm) at a flow rate of 0.6 mL/min with detection by ELSD. The ingredients were identified and quantified using a HPLC library and by comparison with the measured values of standards.

<Evaluation of Solidification>

The obtained powder (dried product of an extracted and purified product of a grape) was taken into a sample bottle and allowed to stand at 25° C. in air with the lid opened. The condition of the sample was observed after 0 hour (i.e., immediately after allowed to stand), 0.25 hours, 0.5 hours, 1 hours, 2 hours and 3 hours and the solidification of the powder due to moisture absorption at each time point was evaluated based on the criteria given below.

A: Powder showed good flowability 1 without solidification B: Powder solidified but made flowable when the bottle was shaken

C: Powder solidified but made flowable when physically crushed

D: Powder solidified with failure to make it flowable

### Example 1

1 kg of the fruit of grapes (Fujinoyume: a cross species between Gyojano-mizu (Vitis flexuosa) and Merlot (Vitis Vinifera)) was crushed with a lab mixer (portable chargeable mixer, model: GN-A01) and added into a beaker (pre-treatment step). As an extractant, 2 L of ethanol with 0.5 mass % 1N hydrochloric acid aqueous solution was then added into the beaker and extraction was effected with stirring for 5 hours at 25° C. (extraction step). The resulting extract was passed through a Celite filter for separation into a supernatant (extract liquid) and a precipitate. The amount of malvidin-3,5-diglucoside in the supernatant was determined by HPLC. The results are shown in Table 2.

### Examples 2-12 and Comparative Examples 1-7

Extract liquids were obtained in the same manner as in Example 1 except that at least one of the variety of grapes, the procedure of the pre-treatment step and the procedure of the extraction step was changed as shown in Table 1. The amounts of malvidin-3,5-diglucoside were measured in the same manner as in Example 1. The results are shown in Table 2.

When the grapes were squeezed in the pre-treatment step, a squeezer (3 L fruit squeezer) was used. In Comparative Example 3, the extractant was adjusted to pH 11 using sodium hydroxide instead of hydrochloric acid.

TABLE 2

| | | | Extractant | | |
|---|---|---|---|---|---|
| | | Pre-treatment | | | Acid |
| | Grape variety | method | Polar solvent | Nonpolar solvent | component |
| Ex. 1 | Fujinoyume | Crush | Ethanol | — | Hydrochloric acid |
| Ex. 2 | Fujinoyume | Squeeze | Water | — | Hydrochloric acid |
| Ex. 3 | Fujinoyume | Squeeze | 50% ethanol | — | Hydrochloric acid |
| | | | aqueous solution | | |
| Ex. 4 | Fujinoyume | Squeeze | Ethanol | — | Hydrochloric acid |
| Ex. 5 | Vitis Coignetiae | Crush | Methanol | — | Hydrochloric acid |
| Ex. 6 | Fujinoyume | Squeeze | Methanol | — | Formic acid |
| Ex. 7 | Vitis Coignetiae | Squeeze | Dimethylformamide | — | Hydrochloric acid |
| Ex. 8 | Fujinoyume | Squeeze | Dimethyl sulfoxide | — | Hydrochloric acid |
| Ex. 9 | Fujinoyume | Crush | Tetrahydrofuran | — | Hydrochloric acid |
| Ex. 10 | Fujinoyume | Squeeze | Ethanol | — | Hydrochloric acid |
| Ex. 11 | Fujinoyume | Squeeze | Ethanol | — | Citric acid |
| Ex. 12 | Fujinoyume | Squeeze | Ethanol | — | Hydrochloric acid |
| Comp. Ex. 1 | Fujinoyume | Squeeze | — | Chloroform | Hydrochloric acid |
| Comp. Ex. 2 | Fujinoyume | Squeeze | — | Toluene | Hydrochloric acid |
| Comp. Ex. 3 | Fujinoyume | Squeeze | Water | — | Adjusted to pH 11 |
| | | | | | with NaOH |
| Comp. Ex. 4 | Fujinoyume | Squeeze | Ethanol | — | Hydrochloric acid |
| Comp. Ex. 5 | Fujinoyume | Squeeze | Ethanol | — | Hydrochloric acid |
| Comp. Ex. 6 | Fujinoyume | Crush | Ethanol | — | Hydrochloric acid |
| Comp. Ex. 7 | Fujinoyume | Squeeze | Ethanol | — | Hydrochloric acid |

| | Extractant | | Malvidin-3,5-diglucoside |
|---|---|---|---|
| | Acid component conc. | Extraction temp. | content |
| | [mol/L] | [° C.] | [mg/1 kg grape fruit] |
| Ex. 1 | 0.005 | 25 | 1640 |
| Ex. 2 | 0.0001 | 30 | 1010 |
| Ex. 3 | 0.01 | 25 | 1170 |
| Ex. 4 | 0.01 | 20 | 1290 |
| Ex. 5 | 0.001 | 25 | 1030 |
| Ex. 6 | 0.01 | 25 | 1170 |
| Ex. 7 | 0.01 | 20 | 1010 |
| Ex. 8 | 0.005 | 25 | 1040 |
| Ex. 9 | 0.00001 | 30 | 1020 |
| Ex. 10 | 0.1 | 25 | 1190 |
| Ex. 11 | 0.0001 | 25 | 1100 |
| Ex. 12 | 0.01 | 45 | 1400 |
| Comp. Ex. 1 | 0.01 | 25 | Not Detected |
| Comp. Ex. 2 | 0.01 | 25 | Not Detected |
| Comp. Ex. 3 | Adjusted to pH 11 | 25 | Not Detected |
| | with NaOH | | |
| Comp. Ex. 4 | 0.005 | 0 | 690 |
| Comp. Ex. 5 | 0.001 | 10 | 810 |
| Comp. Ex. 6 | 0.01 | 60 | 800 |
| Comp. Ex. 7 | 0.01 | 90 | 750 |

It can be seen from Table 2 that Examples 1 to 12 were able to extract malvidin-3,5-diglucoside from grapes in higher yields as compared to Comparative Examples 1 and 2 wherein nonpolar solvents were used, Comparative Example 3 wherein extraction was performed under alkaline conditions, Comparative Examples 4 and 5 wherein extraction was performed at low temperatures, and Comparative Examples 6 and 7 wherein extraction was performed at high temperatures.

### Example 13

The supernatant (extract liquid) obtained in Example 1 was concentrated under reduced pressure to a volume of about 1 L at 50° C. or lower.

One volume of water was then added and the mixture further concentrated under reduced pressure at 50° C. or lower to afford 1 L of an aqueous grape extract solution.

300 mL of the aqueous grape extract solution thus obtained was passed through an open column packed with 40 mL of a synthetic adsorption resin (trade name: DIAION HP20, manufactured by Mitsubishi Corporation) at a temperature of 25° C. and a space velocity (SV) of 5 h⁻¹ (step (a)). Thereafter, an aqueous hydrochloric acid solution adjusted to pH 3 was passed through the open column at a temperature of 25° C. for washing to remove non-adsorbed components (step (b)). 300 mL of ethanol with 0.5 mass % 1N hydrochloric acid aqueous solution was then passed through the open column at a temperature of 25° C. and a space velocity (SV) of 5 h⁻¹ to afford an ethanol fraction (extracted and purified product of a grape) (step (c)). The ethanol fraction was concentrated under reduced pressure and lyophilized to afford a powder (dried product of an extracted and purified product of a grape).

The dry mass of the powder thus obtained was measured in a nitrogen atmosphere, followed by quantification by high-performance liquid chromatography (HPLC) and evaluation of solidification. The results are shown in Table 3.

### Example 14

An aqueous grape extract solution was obtained in the same manner as in Example 13.

To a beaker containing 50 mL of the aqueous grape extract solution was added 20 mL of a synthetic adsorption resin (trade name: Purolite PAD900, manufactured by Purolite Corporation) and stirred at 25° C. for 5 hours (step (a)). The supernatant and the synthetic adsorption resin were separated by suction filtration and the synthetic adsorption resin was washed with 100 mL of an aqueous hydrochloric acid solution (pH 3) to remove non-adsorbed components (step (b)). Next, the synthetic adsorption resin was added to 50 mL of ethanol with 0.5 mass % 1N hydrochloric acid aqueous solution and the mixture stirred at 25° C. for 2 hours (step (c)). The supernatant and the synthetic adsorption resin were separated by suction filtration, 50 mL of water was added to the supernatant, and the mixture was concentrated under reduced pressure to a volume of 50 mL and lyophilized to afford a powder (dried product of a purified and extracted product of a grape).

The dry mass of the powder thus obtained was measured in a nitrogen atmosphere, followed by quantification by high-performance liquid chromatography (HPLC) and evaluation of solidification. The results are shown in Table 3.

### Examples 15 to 19

Powders (dried products of extracted and purified products of grapes) were obtained in the same manner as in Example 13 or Example 14 except that the working conditions of step (a) to step (c) were changed as shown in Table 3.

The dry mass of each powder thus obtained was measured in a nitrogen atmosphere, followed by quantification by high-performance liquid chromatography (HPLC) and evaluation of solidification. The results are shown in Table 3.

### Comparative Example 8

An aqueous grape extract solution was obtained in the same manner as in Example 13.

100 mL of the aqueous grape extract solution was concentrated under reduced pressure and lyophilized as it is to afford a powder.

The dry mass of the powder thus obtained was measured in a nitrogen atmosphere, followed by quantification by high-performance liquid chromatography (HPLC) and evaluation of solidification. The results are shown in Table 3.

TABLE 3

| | | | | Amount of | | |
|---|---|---|---|---|---|---|
| | | | Purification | aqueous grape | Dry powder | |
| | Purification | | temp. | extract solution | Amount | Malvidin-3,5-diglucoside |
| | process type | Resin | [° C.] | [g] | obtained [g] | content [%] |
| Ex. 13 | Column | Synthetic adsorption resin | 25 | 300 | 1.8 | 32 |
| | | DIAION HP20 | | | | |
| Ex. 14 | Batch | Synthetic adsorption resin | 25 | 50 | 0.5 | 12 |
| | | Purolite PAD900 | | | | |
| Ex. 15 | Column | Synthetic adsorption resin | 50 | 50 | 0.4 | 25 |
| | | Purolite PAD550 | | | | |
| Ex. 16 | Batch | Synthetic adsorption resin | 10 | 50 | 0.4 | 21 |
| | | Purolite PAD550 | | | | |
| Ex. 17 | Batch | Cation exchange resin | 25 | 50 | 0.1 | 7 |
| | | DIAION WK10 | | | | |
| Ex. 18 | Batch | Synthetic adsorption resin | 0 | 50 | 0.1 | 9 |
| | | Purolite PAD900 | | | | |
| Ex. 19 | Batch | Synthetic adsorption resin | 70 | 50 | 0.2 | 6 |
| | | Purolite HP20 | | | | |
| Comp. Ex. 8 | — | — | — | 100 | 4 | 2 |

| | Dry powder | | | | | | | |
|---|---|---|---|---|---|---|---|---|
| | Fructose | Glucose | Evaluation of solidification | | | | | |
| | content [%] | content [%] | 0 h | 0.25 h | 0.5 h | 1 h | 2 h | 3 h |
| Ex. 13 | ≤2 | ≤2 | A | A | A | A | A | A |
| Ex. 14 | 5 | 9 | A | A | A | A | A | A |
| Ex. 15 | 17 | 16 | A | A | A | A | A | B |
| Ex. 16 | 22 | 25 | A | A | A | A | B | B |
| Ex. 17 | 36 | 38 | B | C | D | D | D | D |
| Ex. 18 | 39 | 32 | B | C | C | D | D | D |
| Ex. 19 | 37 | 36 | C | C | D | D | D | D |
| Comp. Ex. 8 | 42 | 42 | C | D | D | D | D | D |

It can be seen from Table 3 that Examples 13 to 19 wherein purification was performed were able to reduce the saccharide content while increasing the malvidin-3,5-diglucoside content and produce a powder that does not easily solidify as compared to Comparative Example 8 wherein no purification was performed. It can also be seen from Table 3 that Examples 13 to 16 were able to further reduce the saccharide content while further increasing the malvidin-3,5-diglucoside content and produce a powder that does not more easily solidify as compared to Example 17 wherein a cation exchange resin was used, Example 18 wherein the purification temperature was low, and Example 19 wherein the purification temperature was high.

### INDUSTRIAL APPLICABILITY

According to the disclosed method for producing a grape extract liquid, it is possible to extract malvidin-3,5-diglucoside from a grape in high yields. According to the disclosed method for producing an extracted and purified product of a grape, the disclosed method for producing a dried product of an extracted and purified product of a grape, and the disclosed grape extract, it is possible to provide a grape extract that, even when converted into dry powder form, does not easily solidify and has excellent handleability.

1. A grape extract comprising malvidin-3,5-diglucoside and a saccharide,
wherein a malvidin-3,5-diglucoside content in dry mass of the grape extract is 3% by mass or more, and
a saccharide content in dry mass of the grape extract is 80% by mass or less.

2. A method for producing a grape extract liquid, comprising extracting a grape at an extraction temperature of 15° C. or higher and 50° C. or lower using an extractant containing a polar solvent and an acid component.

3. The method according to claim 2, wherein the polar solvent comprises at least one solvent selected from the group consisting of water, methanol, ethanol, isopropanol, butanol, propylene glycol, butylene glycol, glycerin, acetone, ethyl acetate, methyl ethyl ketone, dimethylformamide, dimethyl sulfoxide, and tetrahydrofuran.

4. The method according to claim 2, wherein the extractant comprises at least one acid selected from the group consisting of formic acid, citric acid, lactic acid, malic acid, tartaric acid, succinic acid, hydrochloric acid, and phosphoric acid.

5. The method according to claim 2, wherein the grape comprises a pericarp of one or more types of grapes selected from the group consisting of Vitis Coignetiae, Vitis flexuosa, a cross species between Vitis Coignetiae and Vitis flexuosa, a cross species between Vitis Coignetiae and Vitis Vinifera, and a cross species between Vitis flexuosa and Vitis Vinifera.

6. The method according to claim 2, wherein the grape is crushed or squeezed.

7. A method for producing an extracted and purified product of a grape, comprising purifying a grape extract liquid produced using the method according to claim 2.

8. The method according to claim 7, wherein the purifying comprises:
a step (a) of bringing the grape extract liquid in contact with a resin;
a step (b) of washing the resin with an acidic aqueous solution after step (a); and
a step (c) of bringing the resin in contact with a mixed liquid of an acidic aqueous solution and an alcohol after step (b).

9. The method according to claim 8, wherein the resin comprises at least one resin selected from the group consisting of an aromatic resin, a substituted aromatic resin, and an acrylic resin.

10. The method according to claim 8, wherein the acidic aqueous solution comprises at least one acid selected from the group consisting of formic acid, citric acid, lactic acid, malic acid, tartaric acid, succinic acid, hydrochloric acid, and phosphoric acid.

11. A method for producing a dried product of an extracted and purified product of a grape, comprising drying an extracted and purified product of a grape produced using the method according to claim 7.
